(19) United States
(12) Patent Application Publication
(10) Pub. No.: US 2004/0144025 A1
(43) Pub. Date: Jul. 29, 2004

(54) Alteration of plant nitrate and oxalic acid concentration

(76) Inventor: Corinne Johnson Rutzke, Ithaca, NY (US)

Correspondence Address: Schwegman Lundberg, Woessner & Kluth, PO Box 2938, Minneapolis, MN, 55402, US

(21) Appl. No.: 10/415,430

(22) PCT Filed: Oct. 5, 2001
(86) PCT No.: PCT/US01/31254

(51) Int. Cl. A01G 13/00; A01G 31/00

(52) U.S. Cl. 47/57.7

(57) ABSTRACT

The present invention provides methods for altering the concentration or level of nitrate, soluble oxalic acid or oxalate, or nutritional quality indicators in a plant, comprising growing the plant in a hydroponic nutrient medium which contains a nitrogen source, and then alterning the content of the hydroponic medium prior to harvest of the plant so that the medium does not contain a nitrogen source, so that the concentration of oxalic acid or oxalage in the plant is altered.

### BACKGROUND OF THE INVENTION

Oxalic acid is an organic acid present in algae, fungi, lichens, higher plants, and animals including humans (Oke, 1969). The role of oxalic acid is unclear and it is usually thought to be a useless end product of carbohydrate metabolism (Hodgkinson, 1977). Oxalic acid forms crystals with several minerals including (but not limited to) calcium, magnesium, potassium, sodium, iron, and zinc. The oxalic acid in spinach is primarily bound to calcium or potassium salts. Oxalic acid levels as high as 13% of the dry weight of spinach have been reported for some cultivars (Hodgkinson, 1977).

Plants, animals and humans form oxalic acid as a secondary metabolite of vitamin C (Hodgkinson, 1977). Human urine always contains small levels of calcium oxalate (Oke, 1969). Deposits of calcium oxalate crystals in the kidneys are a common form of kidney stone (Massey et al, 1993). In healthy adults, with U.S. or European type diets, 90% of oxalic acid excreted in the urine comes from endogenous synthesis (Massey et al., 1993) and is a secondary metabolite of ascorbic acid (vitamin C) in both plants and animals. However, reduction of foods known to increase urinary oxalate is recommended as a dietary change for kidney stone-formers (Massey et al, 1993). High dietary oxalate is associated with reduced calcium absorption from the gut, as oxalate has the ability to bind calcium. Calcium oxalate crystals formed in the gut are not absorbed, but are carried out with the feces. Thus sufficient dietary calcium reduces absorption or oxalate from foods (Massey et al., 1993).

The top 8 foods or plants known to increase urinary oxalate are identified by Massey et al (1993) as rhubarb, spinach, beets, nuts, chocolate, strawberries, wheat bran, and tea. Other foods or plants containing oxalate include leafy green vegetables, asparagus, runner beans, beetroot, brussel sprouts, cabbage, carrots, cauliflower, celery, chives, lettuce, marrow, mushrooms, onions, parsley, green peas, potatoes, radishes, rhubarb, spinach, tomatoes, turnips, apples, apricots, ripe bananas, gooseberries, grapefruits, melons, oranges, peaches, pears, pineapples, plums, blueberries, raspberries, strawberries, arugula, beet greens, collard greens, kale, endive, bok choy, dandelion greens, escarole, cole, mache, mustard greens, radicchio, rapini, swiss chard, and watercress.

There have been cases of oxalate-poisoning, especially from the species rhubarb (Rheum rhaponticum, L.) and sorrel grass (Rumex acetosa. L.). In high levels, oxalic acid can be corrosive to the gastrointestinal tract (Hodgkinson, 1977). Cattle have been poisoned by high oxalate content of Setaria found in grazing areas. However, workers in Australia found a cultivar of Setaria cattle can graze safely (Hodgkinson, 1977).

Oxalic acid oxidase is an enzyme that catalyses the breakdown of oxalic acid into carbon dioxide and hydrogen peroxide. Oxygen is required for the reaction. The formula for the reaction is:

C₂O₄H₂+O₂→(oxalic acid oxidase)→2CO₂+H₂O₂

The enzyme is present in spinach leaves, but is usually not active. Through studies on beet root extracts it was determined that the sole factor inhibiting oxalic acid oxidase was nitrate. Low concentration of nitrate in vitro (50 μM) is sufficient to inhibit the enzyme (Oke, 1969).

Varietal differences in spinach oxalate concentration have been reported.

Differences in oxalate concentration have been reported with respect to plant age. The oxalate concentration, in general, increases with plant age and led to the suggestion that earlier harvesting may be beneficial. However, some investigators found oxalate increased with plant age (Hodgkinson, 1977), others reported spinach oxalate concentration decreases with age.

An enzyme, oxalic acid oxidase, catalyses the break down of oxalic acid into carbon dioxide and hydrogen peroxide in the presence of oxygen. Oxalic acid oxidase has been studied in spinach leaves (Oke, 1969). Although present in the leaves, the oxidase is inhibited by even low levels of nitrate (<−50 M) (Oke, 1969). Nitrate may also play another role in increasing leaf oxalate.

The evolutionary advantage of spinach possessing higher oxalate than most other vegetables has not been determined. Some have suggested it may be protective against foraging animals (Hodgkinson, 1977).

Nitrate is ubiquitous in most fresh vegetables and accounts for approximately 80% of nitrate in the diet. Nitrate itself is not particularly toxic. Most nitrate is excreted from the body through the urine (Lee, 1970). However approximately 5% of consumed nitrate is converted to nitrite in the oral cavity, gastrointestinal tract, and liver. It is the reduced form of nitrate, nitrite, that is the primary cause for concern. High levels of nitrate consumed by infants can lead to methemoglobinaemia, or blue baby syndrome. The primary concern for healthy adults is the potential for post-consumption formation of carcinogenic N-nitrosamine compounds. Fresh spinach may contain up to 740 ppm nitrate, and reducing soluble nitrate and oxalate levels in vegetables would improve the nutritional value of these crops to humans or other animals.

There is a need for altering certain compounds in plants in order to improve the nutritional quality of the plant for human consumption.

### SUMMARY OF THE INVENTION

The present invention provides methods for altering the concentration or level of soluble oxalic acid or oxalate in a plant, comprising growing the plant in a hydroponic nutrient medium which contains a nitrogen source, and then altering the content of the hydroponic medium prior to harvest of the plant so that the medium does not contain a nitrogen source, so that the concentration of oxalic acid or oxalate in the plant is altered.

The methods of the present invention may also be used to alter the concentration or level of soluble nitrate in a plant, and to alter the concentration or level of nitrate and soluble oxalic acid or oxalate in a plant. Further, the methods of the present invention may be used to alter the concentration or level of one or more nutrient quality indicators in a plant.

### BRIEF DESCRIPTION OF THE FIGURES

FIG. 1 Oxalate and Nitrate concentration in spinach leaves treated with six pre-harvest draw-down method treatments.

FIG. 2 Time-course pre-harvest draw down of nitrate and oxalate in spinach, cv Whitney transferred to RO water for five days.

FIG. 3 Time course reduction in nitrate and oxalate in spinach cv Whitney transferred to RO water for 192 h (8 days).

FIG. 4 Comparison of spinach treated for 192 h (8 d) in RO water, RO water+tent, or control plants in a nitrate nutrient solution.

FIG. 5 Reduction in nitrate and oxalate in spinach cv Alrite transferred to RO water for 168 h (7 days). Spinach treated in RO water, RO water+tent, or control plants in a nitrate nutrient solution.

FIG. 6 Comparison of spinach cv Whitney treated for 192 h (8 d) in TAP water, TAP water+tent, or control plants in a nitrate nutrient solution.

FIG. 7 Time course reduction in nitrate and oxalate in spinach cv Whitney transferred to TAP water for up to 264 h (11 days).

FIG. 8 Combined data points from all six oxalate draw-down experiments show similarity in slope of lines during draw-down regardless of starting concentration, tap or RO water, and spinach cultivar Whitney or Alrite.

FIG. 9 Line of best fit developed from combining oxalate draw down data from six experiments.

FIG. 10 Combined nitrate concentration data points from Four draw-down experiments show similarity in slope and shape of lines during draw-down. Graph combines results from tap water, RO water, and spinach cultivars Whitney and Alrite.

FIG. 11 Line of best fit developed from combining nitrate draw down data from three experiments.

FIG. 12 During draw-down procedure, nitrate is used in vacuoles and plants can continue to grow (add dry weight).

FIG. 13 Decline in three indicator vitamins with increasing duration of oxalate draw-down period: A) vitamin C, B) Folate, C) Beta-carotene.

FIG. 14 Oxalate concentration in spinach leaves as a function of nitrate concentration. Data from four nitrate draw-down experiments combined shows saturation of nitrate influence on oxalate concentration at 200 umoles/g DW in this set of experiments.

### DETAILED DESCRIPTION OF THE INVENTION

The term Controlled Environment Agriculture (or “CEA”), as is used herein, is a combination of horticultural and engineering techniques that optimize crop production, crop quality, and production efficiency (L. D. Albright, 1990, Environment Control for Animals and Plants).

The term hydroponic nutrient solution or medium, as is used herein, is a water-based formulation containing essential nutrients for plant growth. The solutions contain mineral elements and compounds, containing but not limited to nitrogen, phosphorous, potassium, calcium, magnesium, oxygen, sulphur, boron, chlorine, copper, iron, manganese, molybdenum, zinc, in addition to many minerals present in trace amounts. The pH of the solution is controlled to allow solubility of the mineral elements and compounds in the solution. (Jones, 1997, Hydroponics, A practical Guide for the Soilless Grower). The nitrogen concentration of the hydroponic nutrient solution or medium may range from, but is not limited to, slightly under 50 to more than 350 ppm.

The reduction in concentration of oxalic acid, nitrate, or nutrient quality indicators, as accomplished by the present invention, may be allowed to continue as long as plants maintain a marketable or healthy appearance, or a dark-green appearance.

The term “nutrient quality indicators” includes vitamins and/or minerals in a plant that are important for human or animal nutrition, and includes, but is not limited to vitamin C, ascorbic acid, folate, folic acid, vitamin B9, β-carotene, a vitamin A precursor, lutein, calcium, iron, vitamin A, vitamin E, and other vitamins and minerals.

The term “mature plant,” as is used in this application, includes plants that have reached a sufficient stage of growth and contain the desired characteristics, so that they may be harvested for any useful purpose. As an example, for spinach, the term “mature plant” may describe a plant that has reached approximately 2-7 ounces of fresh weight after about 20-35 days from seeding.

The present invention may be useful in altering the concentrations of oxalic acid, oxalate, nitrate, or nutritional quality indicators of any plant species, including, but not limited to, plants that contain oxalic acid or nitrate, such as spinach, beets, nuts, chocolate, cacao, strawberries, wheat bran, tea, leafy green vegetables, asparagus, runner beans, beetroot, brussel sprouts, cabbage, carrots, cauliflower, celery, chives, duckweed, lettuce, marrow, mushrooms, onions, parsley, green peas, potatoes, radishes, rhubarb, spinach, tomatoes, turnips, apples, apricots, ripe bananas, gooseberries, grapefruits, melons, oranges, peaches, pears, pineapples, plums, blueberries, raspberries, strawberries, arugula, beet greens, collard greens, kale, endive, bok choy, dandelion greens, escarole, cole, mache, mustard greens, radicchio, rapini, swiss chard, and watercress.

The present invention may also be useful in altering the concentrations of oxalic acid, oxalate, nitrate, or nutritional quality indicators of other plant species, including, but not limited to, corn, rapeseed, alfalfa, rice, rye, millet, pearl millet, proso, foxtail millet, finger millet, sunflower, safflower, wheat, soybean, tobacco, potato, peanuts, cotton, potato, cassava, coffee, coconut, pineapple, cocoa, banana, avocado, fig, guava, mango, olive, papaya, cashew, macadamia, almond, sugar beets, sugarcane, oats, duckweed, barley, vegetables, ornamentals, conifers, tomatoes, green beans, lima beans, peas, Cucumis (including cucumber, cantaloupe, and musk melon), azalea, hydrangea, hibiscus, roses, tulips, daffodils, petunias, carnation, poinsettia, chrysanthemum, legumes (including peas and beans, such as guar, locust bean, fenugreek, soybean, garden beans, cowpea, mungbean, lima bean, fava bean, lentils, chickpea), peanuts, crown vetch, hairy vetch, adzuki bean, mung bean, and chickpea, lupine, trifolium, field bean, clover, Lotus, trefoil, lens, lentil, false indigo, alfalfa, orchard grass, tall fescue, perennial ryegrass, creeping bent grass, redtop grass, aneth, artichoke, blackberry, canola, cilantro, clementines, eucalyptus, fennel, grapefruit, honey dew, jicama, kiwifruit, lemon, lime, mushroom, nut, okra, orange, parsley, persimmon, plantain, pomegranate, poplar, radiata pine, Southern pine, sweetgum, tangerine, triticale, vine, yams, apple, pear, quince, cherry, apricot, melon, hemp, buckwheat, grape, raspberry, chenopodium, blueberry, nectarine, peach, plum, watermelon, eggplant, pepper, cauliflower, broccoli, onion, carrot, leek, beet, broad bean, celery, radish, pumpkin, endive, gourd, garlic, snapbean, squash, turnip, asparagas, and zuchini.

### EXAMPLES

Pre-Sowing Seed Treatment

Spinach seeds, cv Whitney, were treated using a modified-Katzman protocol in Experiments I, II, IV and V and VI. Experiment III was performed using the spinach cultivar Alrite. Calcium hypochlorite was removed from the pre-sowing treatment to avoid seed contact with potential sources of chloride ions. Seeds were placed in a 2 liter beaker containing glass distilled water and were stirred in the room temperature water bath for 4 hours with a magnetic stir plate. Water was drained from the beaker and replaced with a 0.3% H₂O₂ solution. Stirring continued overnight. After 15 hours, seeds were removed from packets and placed on blotter paper in germination boxes. Seeds were spread evenly on the blotter paper and distilled water was added to keep germinating seeds moistened. Seeds in germination boxes were then placed in a 5° C. cooler for 7 days while the imbibition and germination continued.

Seed Planting and Hydroponic Pond System Seed Holders

### EXPERIMENT I

Floating polystyrene trays were prepared by cutting eleven 28 cm×0.6 cm (11×0.25 in) strips out of each 0.3 m×0.6 m (1×2 ft) tray. Thirty felt strips were cut from a bolt into rectangles 28 cm×10 cm (11×4 in). Two pieces of felt were placed together to form cloth sandwich-strips and were fed through the polystyrene strip-holders. Felt-strip pairs were held in place by wedging a small piece of polystyrene at both ends of each felt-strip pair. The felt-strip seed holders wicked up water and nutrients from the pond solution and maintained a moist environment around the seeds and roots. The foam boards were 2.5 cm (1 inch) thick and roots grew downward, between the two felt strips into the oxygenated hydroponic solution below. Seeds were placed with root oriented downward between two pieces of felt and held by friction fit over the hydroponic nutrient solution. Only seeds that had germinated during the one-week in the cooler were planted. Three seeds were planted between each felt strip pair. Seedling radicals were approximately 2 to 5 mm long at time of sowing.

### EXPERIMENTS II-V

Seeds were sown into reverse-osmosis (RO) water-leached rockwool flats. The moistened seeded rockwool flats were covered and maintained at 18° C. for 3 days after sowing without need for additional watering. At age 3 days, growth chamber temperature was increased to 22° C. and ebb and flow watering was initiated at a rate of twice per day. Seedlings were watered automatically with a nutrient solution containing no ammonium and no chloride. Cool white fluorescent lamps in the growth chamber were set to 25% from initiation of imbibition to age 3 days. At age 4 days, lights were increased to 50% maximum intensity. At age 12 days after planting seeds in rockwool, Whitney seedlings in rockwool cubes were transferred from the growth chamber to polystyrene floaters in an 18° C. hydroponic pond.

Greenhouse Environmental Parameters

Greenhouse temperature was controlled at 24° C. continuous. Light control program was set to deliver 16 mols of light per day. Supplemental light was provided by an array of high-pressure sodium lamps.

Culture Phase Hydroponic Pond Conditions

The pond solution nutrient medium (or hydroponic nutrient medium) recipe was adjusted to eliminate sources of ammonium and chloride, due to reported toxicity problems in spinach with these ions (Elia et al, 1999). Pond temperature was maintained at 18° C. Nutrient solution pH and electrical conductivity (EC) were monitored and corrections made as needed. Set point for pH was 5.8. Nitric acid was used to decrease pH, and potassium hydroxide was used to increase pH. Electrical conductivity set point was 920 micro S for this particular nutrient solution. Daily water samples were taken and frozen for nitrate analysis. Nitrate concentration in the NO₃ solution ranged from 0.2 to 2.5 mM. Nitrate concentration in the urea pond ranged from 0 to 1.1 mM. Nitrate in the RO water pond ranged from 0 to 0.01 mM.

Nitrate Draw-Down Phase Hydroponic Pond Conditions

### EXPERIMENT I

At age 21 days after sowing, the seedlings in felt strips were divided into three groups. Plants from groups 1 and 2 were transferred to a pond filled with RO water (no nutrient solution). Plants from group 3 (control) remained in the culture nutrient solution. Pond temperature for both ponds was 18° C. Plants were maintained in this condition for 5 days.

### EXPERIMENTS II-VI

At plant maturity (approximate age 28 days) plants were transferred to one of three RO water-filled tanks for 5 days to draw down nitrate in cell vacuoles. Plant from experiments V and VI used tap water as RO water line was not functioning in greenhouse (GH). Tanks were not oxygenated, but were stirred regularly by hand to aerate. Tanks were located in GH 15 C with photoperiod from 6 am to 10 pm, continuous HPS lighting. Greenhouse temperature during nitrate draw-down phase was 20° C. There were three tanks used in the test, with six plants per tank.

Oxalate Draw-Down Phase Hydroponic Pond Conditions

### EXPERIMENT I

After 5 days in the nitrate-draw down pond or control pond, plants were divided into 6 groups. The following list describes the PRE-HARVEST treatment conditions:

1) RO water [symbol] RO water open air

2) RO water [symbol] RO water, inside CO₂ draw-down tent

3) RO water [symbol] Urea solution, open air

4) RO water [symbol] Urea solution, inside CO₂ draw-down tent

5) Nutrient solution [symbol] Nutrient solution open air

6) Nutrient solution [symbol] Nutrient solution, inside CO₂ draw-down tent

Each CO2 draw-down tent was fabricated by placing clear plastic bags over a wire-mesh cage. A beaker containing 150 ml of 1 N KOH with fluted germination paper fan was placed inside each CO₂ draw-down tent. There was no humidity control in the tents for Experiment I.

### EXPERIMENTS II-VI

Six plants in each tank were divided in two. Three plants were placed in a CO₂ draw-down tent. Humidity in tent was controlled with a de-humidifier. A 4 liter open reservoir of 1 N KOH was placed inside the tent to assist in draw down of CO₂. The other three plants in each tank remained outside the tents. Oxalate draw-down time was two days. Tanks were located in GH 15° C. with photoperiod from 6 am to 10 pm, continuous HPS lighting. Greenhouse temperature during oxalate draw-down phase was 20° C.

Harvest

After 8 days in pre-harvest treatment, plants were harvested by separating above-root portion from roots with a razor. Plants were placed in a 70° C. drying oven inside brown paper bags and dried for two days. Plants in the second tap water experiment were freeze dried rather than oven dried for additional nutrient analyses.

Tissue Preparation

Dried plant samples were bulked by treatment and age at harvest and were ground on a Wiley Mill, using a #20 screen.

Chemical Analysis of Oxalate and Nitrate Contents

Soluble oxalate and nitrate were extracted together in the same supernatant. A sample of ground tissue was weighed into a 15 ml tube. Ten milliliters of HCl (0.1 N) was added and mixed on a vortex mixer. Test tubes were sealed and placed on a shaker for one hour at room temperature. Samples were centrifuged and 2 ml of supernatant removed and centrifuged again in 2.2 ml microcentrifuge tubes. A 0.5 ml portion was removed and diluted with 4.5 ml of 18 M ohm water. A 500 microliter aliquot of the diluted sample was placed in a Dionex autosampler vial (0.5 ml vial, Dionex Corp., Sunnyvale, Calif.) for each spinach cultivar.

Chemical Analyses of Vitamin C, Folate, and β-Carotene—Experiment VI Only

To determine whether the draw-down technique was also reducing vitamin content of the spinach, freeze dried tissue samples from three time points during the second tap-water draw-down experiment were sent to ENI Laboratories (www.eurofins.com, Eurofins Scientific, Dayton. N.J.) for accredited nutrient analysis. The time points selected for analysis were Day 0 (starting point control), Day 5 (day nitrate reserves are depleted), and Day 8 (last day spinach had marketable appearance). The number of samples and types of analysis were limited due to cost. Three nutrients were selected as indicators of potential loss of nutrients with the pre-harvest nitrate/oxalate reduction technique. The water soluble vitamin, vitamin C was selected as the first indicator because spinach contains ample amounts of vitamin C for measurement, and ascorbic acid decline with post-harvest food preparation is well documented. Folic acid (total vitamin B9) was selected because spinach is among the vegetables high in folic acid and this would be an important nutrient to preserve. The vitamin A precursor, β-carotene, was selected to represent fat-soluble vitamins.

Statistical Analysis

Statistical analysis was performed using Minitab software (V12, Minitab Inc., State College, Pa.), Microsoft EXCEL 97 (Microsoft Corp, USA) and SigmaPlot software (SigmaPlot 2000, V6.X, SPSS Inc., Chicago, Ill.).

### Results and Discussion

Soluble Nitrate and Oxalic Acid Reduction

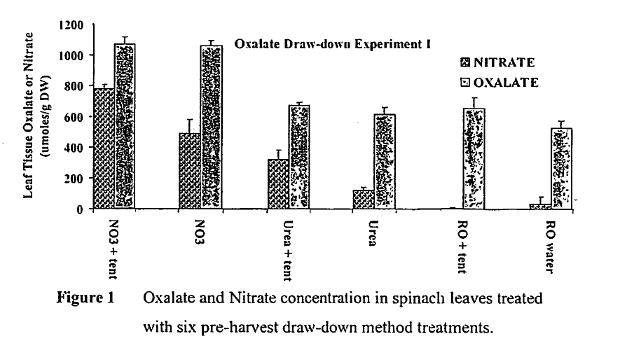

The practice of pre-harvest transfer of spinach plants into RO water and tap water reduced nitrate concentration. In Experiment I, plants in CO₂ draw-down-tents with either NO₃ nutrient solution or urea nutrient solution had higher levels of nitrate than plants in nutrient solutions outside of the tents (FIG. 1). This increase in nitrate concentration was probably due to lowered light levels under the plastic tents.

Transfer of the seedlings into RO water (without tent) was the most effective treatment for reduction of nitrate and oxalate within 8 days. Plants in the nitrate solution (Control) had an average leaf concentration of 1063 μmol oxalate/g dry weight (DW) (10%). Plants in the RO water solution had 655 μmol oxalate/g DW (6%). Nitrate levels were 484 μmol/g DW (4%) in the control plants, and 34 μmol/g DW (>1%) in the RO water treated plants (FIG. 1). From a qualitative standpoint, there were no obvious changes in plant appearance between the treatments. To determine whether this time could be shortened, Experiments II-VI were designed to examine time-course patterns of reduction.

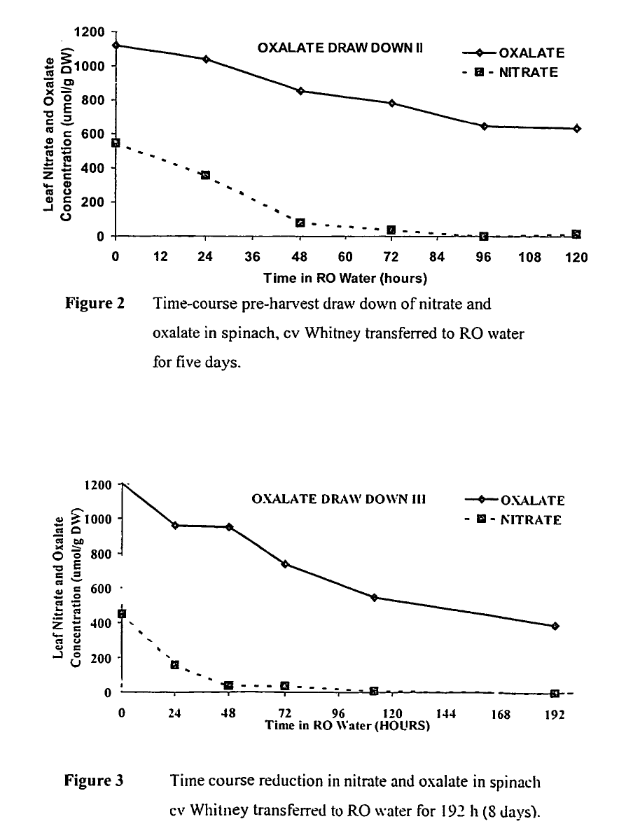

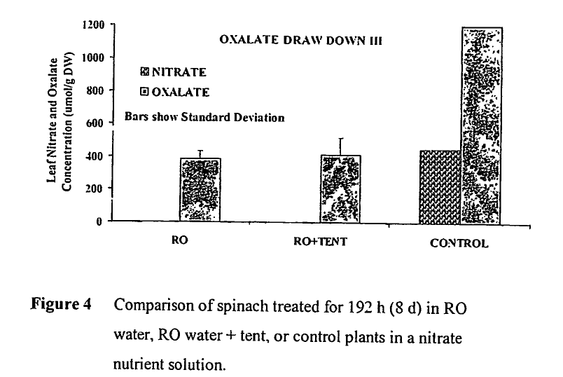

In Experiment II, oxalate was reduced from 1120 to 633 μmol/g DW (i.e., from 10% to 6% DW) in five days of RO water treatment. Nitrate was reduced from 548 to 15 μmol/g DW (i.e., 3% to 0% DW) in five days (120 hours) (FIG. 2). The experiment was extended to eight days in Experiment III, and a humidity-controlled tent treatment was added as an end-point for comparision with controls. For the time-course portion of Experiment III, oxalate concentration was reduced from 1203 to 387 μmol/g DW (i.e., from 11% to 3% DW) in eight days (FIG. 3). Nitrate concentration was reduced from 449 to 0 μmol/g DW (i.e., 3% to 0% DW) in eight days. The RO water+tent treatment plants were not significantly different from the plants given the RO water treatment alone (FIG. 4).

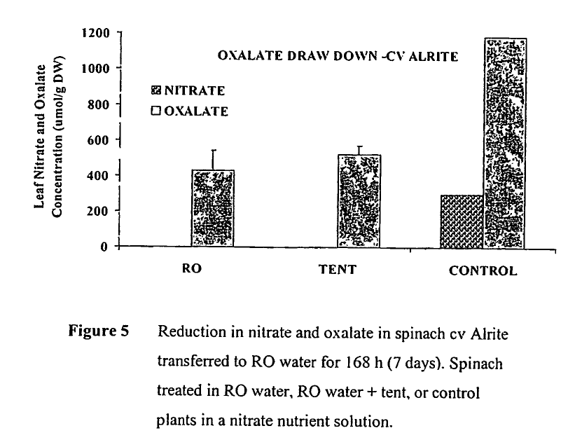

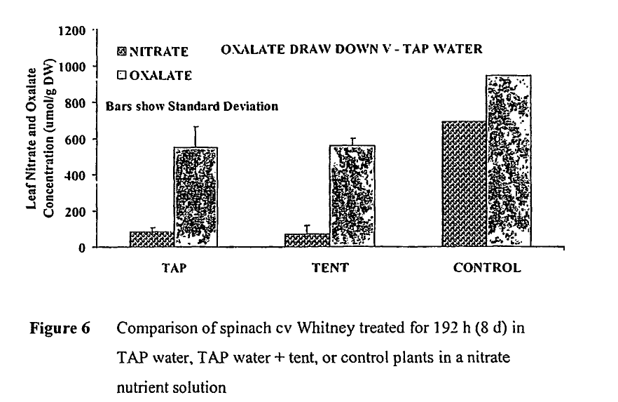

The same procedure was performed for seven days on another cultivar of spinach, Alrite (FIG. 5). Alrite has a faster bolting tendency than Whitney. Alrite leaf nitrate concentration was reduced from 299 to 0 μmol/ g DW (i.e., from 2% to 0% DW) and leaf oxalate concentration was reduced from 1181 to 432 μmol/g DW (i.e., from 10% to 4% DW) after 7 days of the draw-down treatment in RO water (FIG. 5). Soluble leaf nitrate content was lower in Alrite (299 μmol/g DW) than in Whitney (449 μmol/g DW). The lower concentration of nitrate in Alrite than in Whitney is consistant with findings in other experiments (e.g., Table 4.4) and may be a genetic difference in leaf nitrate concentration. The results of the oxalate draw-down procedure were the same for Alrite as for Whitney.

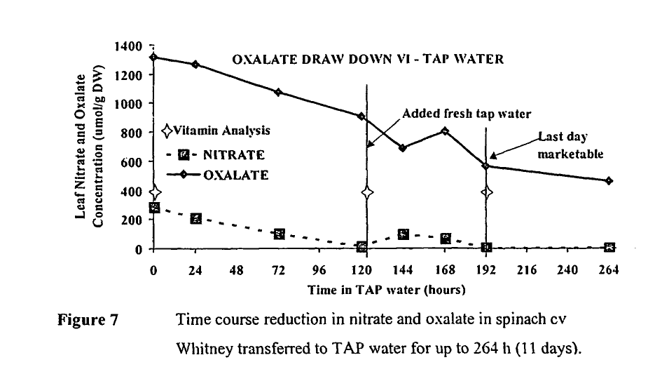

In Experiment V, the draw-down method was performed using tap-water instead of RO water to determine whether cost could be lowered by using tap water for the method. Nitrate was 691 μmol/g DW (4% DW) in the control treatment, and 83 μmol/g DW (1% DW) in the tap water treament. Tap water contains nitrate, so it is not surprising that the tap water treated plants did not reach 0 nitrate in the same time that the RO water treated plants reached 0 nitrate concentration. Oxalate was 945 μmol/g DW (8% DW) in the control plants and 552 μmol/g DW (5% DW) in tap-water treated plants (FIG. 7).

The final draw-down test, Experiment VI, was a time-course sampling with plants treated in tap water. Three samples were also analyzed for three nutrient indicators (vitamin analysis—next section). Oxalate levels were reduced from 1317 to 461 μmol/g DW (i.e., from 12% to 4%, DW). The plants were yellowed on the last harvest day (264 h, or day 11) and would not have been marketable. Daily qualitative observations recorded note that day 8 (192 h) was the last day the plants appeared healthy and marketable. Leaf oxalate concentration on day 8 (192 h) was 565 μmol/g DW (5% DW). Leaf nitrate concentration was reduced from 284 to 0 μmol/g DW (2% to 0% DW). Fresh tap water was added to the treatment ponds after to 120 h harvest (day 5). Tap water contains nitrate, and the increase in pond-water nitrate due to adding fresh water resulted in leaf concentration increasing on day 6 (144 h) and then falling again within 2 days. Interestingly, when leaf nitrate concentration rose, leaf oxalate concentration followed and increased one day after the nitrate peak (day 7) at 168 hours. It is notable that the increase in oxalate following the water-refresh did not shift the draw-down to a higher recovery level. FIG. 7 shows nitrate and oxalate leaf concentrations during the course of the tap-water draw-down method performed on spinach, cv Whitney. Also indicated are the three sampling dates for vitamin Analysis. The last marketable day is indicated, but oxalate concentration continued to fall until the last plants were removed on day 11 (264 h).

Use of RO water was superior to tap when water-replenishment was needed due to presence of nitrate in tap water. Oxalic acid was reduced by one-half to two-thirds through the 7 to 8 day pre-harvest treatment. The reduction in oxalic acid paralleled nitrate reduction in time-course studies.

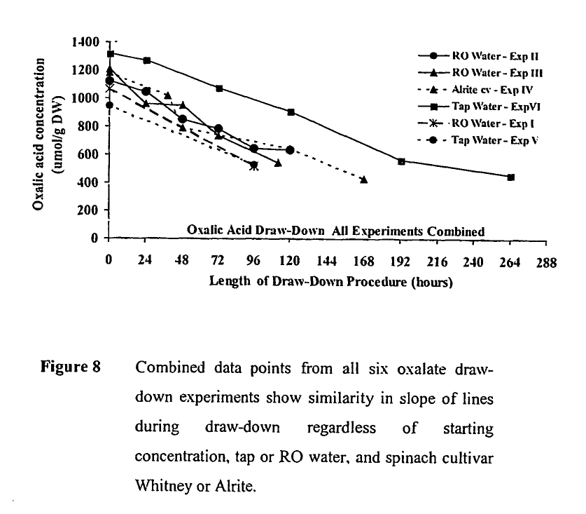

By combining data from all six draw-down experiments, the reduction in soluble oxalate is linear, with a slope that was constant in all experiments, regardless of starting point of soluble oxalic acid concentration in the leaves (FIG. 8).

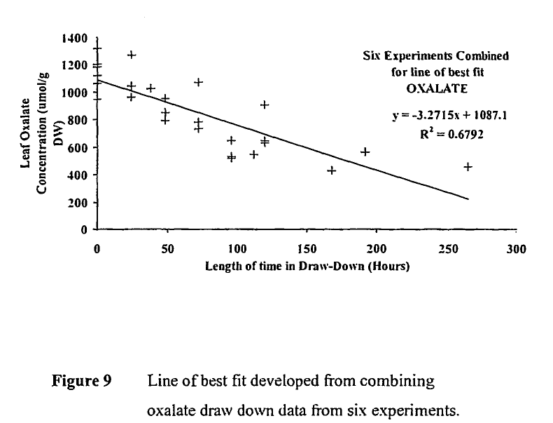

From these data points, a combined line of best fit was calculated by EXCEL (FIG. 9).

From the linear line of best fit for soluble oxalate reduction:

[Oxalate Concentration (μmol/g DW)]=−3.2715*(hrs in treatment)+1087.1

The slope of the line is approximately −3.3 and indicates that in the method developed here, oxalate is removed at a rate of 3.3 umol per hour per g DW. This is equivalent to a removal rate of approximately 0.02% oxalate/g DW per hour. It would be expected then, that 78.5 umol oxalate are removed per day per g DW (i.e., 0.49% DW per day)

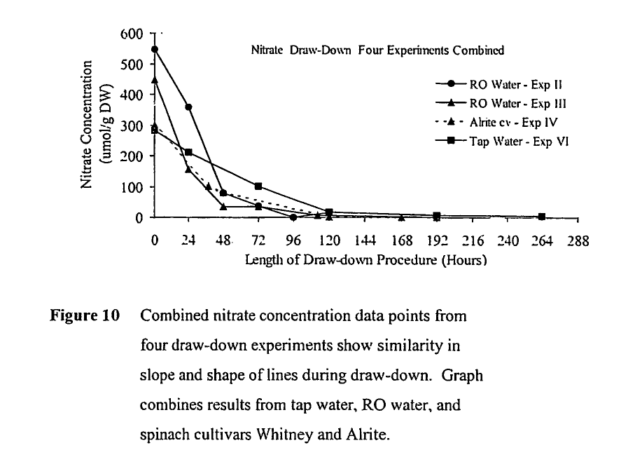

Combining data of the nitrate concentration during draw-down does not show a linear pattern (FIG. 10).

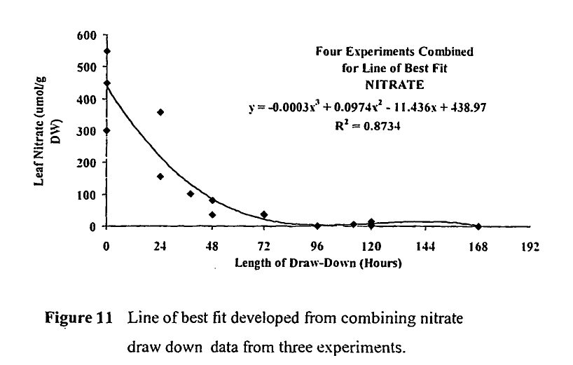

The response to draw-down is not as rapid from the tap water as RO water, as would be expected because tap water contains nitrate. For this reason, only the other three experiment lines are used for the trend-line fitting (FIG. 11)

Plant Growth During Nitrate Draw-Down

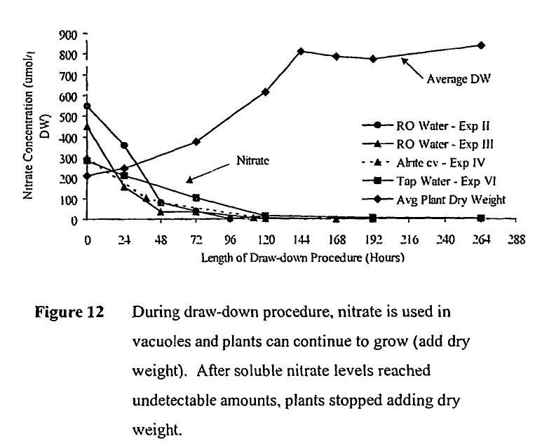

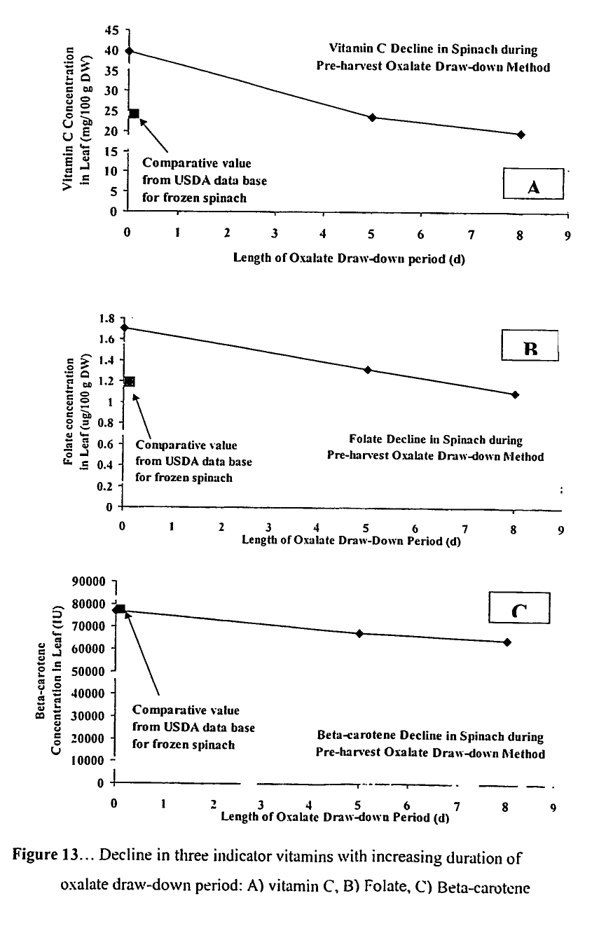

Plant dry weight data from the final draw-down experiment demonstrates that the spinach plants continue to grow during the nitrate draw-down period, as the plants are using up nitrate stores in the vacuoles. When the nitrate reaches 0, plant growth stops (FIG. 12).

Vitamin Reduction with Pre-Harvest Treatment

Nutrient analysis of tissue from days 0, 5, and 8 of the oxalic acid draw-down treatment show that all three indicator vitamins declined with increasing length of oxalate draw-down period (FIG. 13). Vitamin C (ascorbic acid) concentration declined from 39.6 to 19.8 mg/100 g DW in 8 days. Vitamin B9 (Folic acid) concentration in the leaves declined from 1.71 to 1.10 mg/100 g DW during the 8 day period. Vitamin A precursor (Beta-carotene) concentration declined from 76900 to 64300 IU/100 g DW. The linearity of the decline in nutrients is indicated by the r² correlation coefficients of the lines.

For vitamin C decline, r²=0.9611, and equation:

[vitamin C (mg/100 g DW)]=−2.549*(Days of draw-down)+38.712.

For Folate decline, r²=0.9997, and equation:

[Folate (ug/100 g DW)]=−0.0764*(Days of draw-down)+1.7079

For vitamin A precursor, Beta-carotene, r²=0.9776, and equation:

[Beta-carotene (IU/100 g DW)]=−1610.2*(Days of draw-down)+76478

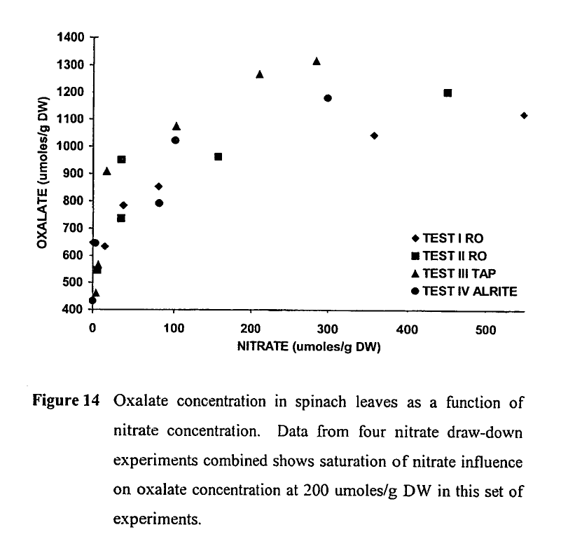

By combining all data points for the six nitrate and oxalate draw-down experiments, the graph of oxalate concentration as a function of nitrate concentration shows a narrow range of response before the influence of increasing nitrate was saturated (FIG. 14).

The pre-harvest technique reduced soluble nitrate levels in the spinach leaves to undetectable levels by day 5 of the pre-harvest draw-down treatment. Soluble oxalic acid also reduced by one-half to two thirds by the method. Reduction in oxalic acid occurred and paralleled the reduction in nitrate in all time-course studies. The leaf concentration of the three vitamin indicators also declined with increasing time in the oxalate draw-down treatment.

### References

All publications, patents and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

In addition, the following references have been cited in the text and their complete citations are as follows:

Elia, A., P. Santamaria, and F. Serio (1998) Nitrogen nutrition, yield and quality of spinach, Journal of the Science of Food and Agriculture 76:341-346.

Hodgkinson, A. (1977) Oxalic Acid in Biology and Medicine, Academic Press, London.

Massey, L. K., H. Roman-Smith, and R. A. L. Sutton (1993) Effect of dietary oxalate and calcium on urinary oxalate and risk of formation of calcium oxalate kidney stones, Journal of the American Dietetic Association 93:901-906.

Oke, O. L. (1969) Oxalic acid and plants and in nutrition, World review of Nutrition and Dietetics 10:262-303.

1. A method for altering the concentration or level of soluble oxalic acid or oxalate in a plant, comprising
a. growing the plant in a hydroponic nutrient medium which contains a nitrogen source; and
b. altering the content of the hydroponic medium prior to harvest of the plant so that the medium does not contain a nitrogen source, so that the concentration of oxalic acid or oxalate in the plant is altered.

2. A method for altering the concentration or level of soluble nitrate in a plant, comprising
a. growing the plant in a hydroponic nutrient medium which contains a nitrogen source; and
b. altering the hydroponic medium prior to harvest of the plant so that the medium does not contain a nitrogen source, so that the nitrate concentration of the plant is altered.

3. A method for altering the concentration or level of nitrate and soluble oxalic acid or oxalate in a plant, comprising
a. growing the plant in a hydroponic nutrient medium which contains a nitrogen source; and
b. altering the hydroponic medium prior to harvest of the plant so that the medium does not contain a nitrogen source, so that concentrations of oxalic acid and nitrate in the plant are altered.

4. A method for altering the concentration or level of one or more nutrient quality indicators in a plant, comprising
a. growing the plant in a hydroponic nutrient medium which contains a nitrogen source; and
b. altering the hydroponic medium prior to harvest of the plant so that the medium does not contain a nitrogen source, so that the concentration of one or more nutrient quality indicators in the plant is altered.

5. A method for reducing the concentration or level of soluble oxalic acid or oxalate in a plant, comprising
a. growing the plant in a hydroponic nutrient medium which contains a nitrogen source; and
b. altering the hydroponic medium prior to harvest of the plant so that the medium does not contain a nitrogen source, so that the concentration of oxalic acid or oxalate in the plant is reduced.

6. A method for reducing the concentration or level of soluble nitrate in a plant, comprising
a. growing the plant in a hydroponic nutrient medium which contains a nitrogen source; and
b. altering the hydroponic medium prior to harvest of the plant so that the medium does not contain a nitrogen source, so that the nitrate concentration of the plant is reduced.

7. A method for reducing the concentration or level of nitrate and soluble oxalic acid or oxalate in a plant, comprising
a. growing the plant in a hydroponic nutrient medium which contains a nitrogen source; and
b. altering the hydroponic medium prior to harvest of the plant so that the medium does not contain a nitrogen source, so that concentrations of oxalic acid and nitrate are reduced in the plant.

8. A method for reducing the concentration or level of one or more nutrient quality indicators in a plant, comprising
a. growing the plant in a hydroponic nutrient medium which contains a nitrogen source; and
b. altering the hydroponic medium prior to harvest of the plant so that the medium does not contain a nitrogen source, so that the concentration of one or more nutrient quality indicators is reduced in the plant.

9. A method for altering the concentration or level of soluble oxalic acid or oxalate in a plant, comprising
a. growing the plant in a hydroponic nutrient medium which contains a nitrogen source; and
b. reducing the source of nitrogen in the medium prior to harvest of the plant so that the oxalic acid concentration in the plant is altered.

10. A method for altering the concentration or level of soluble nitrate in a plant, comprising
a. growing the plant in a hydroponic nutrient medium which contains a nitrogen source; and
b. reducing the source of nitrogen in the medium prior to harvest of the plant so that the nitrate concentration of the plant is altered.

11. A method for altering the concentration or level of nitrate and soluble oxalic acid or oxalate and nitrate in a plant, comprising
a. growing the plant in a hydroponic nutrient medium which contains a nitrogen source; and
b. reducing the source of nitrogen in the hydroponic medium prior to harvest of the plant so that concentrations of oxalic acid and nitrate in the plant are altered.

12. A method for altering the concentration or level of one or more nutrient quality indicators in a plant, comprising
a. growing the plant in a hydroponic nutrient medium which contains a nitrogen source; and
b. reducing the source of nitrogen in the hydroponic medium prior to harvest of the plant so that the concentration of one or more nutrient quality indicators in the plant is altered.

13. A method for reducing the concentration or level of soluble oxalic acid or oxalate in a plant, comprising
a. growing the plant in a hydroponic nutrient medium which contains a nitrogen source; and
b. reducing the source of nitrogen in the hydroponic medium prior to harvest of the plant so that the oxalic acid concentration in the plant is reduced.

14. A method for reducing the concentration or level of soluble nitrate in a plant, comprising
a. growing the plant in a hydroponic nutrient medium which contains a nitrogen source; and
b. reducing the source of nitrogen in the hydroponic medium prior to harvest of the plant so that the nitrate concentration in the plant is reduced.

15. A method for reducing the concentration or level of nitrate and soluble oxalic acid or oxalate in a plant, comprising
a. growing the plant in a hydroponic nutrient medium which contains a nitrogen source; and then
b. reducing the source of nitrogen in the hydroponic medium prior to harvest of the plant so that concentrations of oxalic acid and nitrate in the plant are reduced.

16. A method for reducing the concentration or level of one or more nutrient quality indicators in a plant, comprising
a. growing the plant in a hydroponic nutrient medium which contains a nitrogen source; and
b. reducing the source of nitrogen in the hydroponic medium prior to harvest of the plant so that the concentration of one or more nutrient quality indicators in the plant is reduced.

17. The method as in any of claims 1-16, wherein the plant is selected from the group consisting of rhubarb, spinach, beets, nuts, chocolate, strawberries, wheat, and tea.

18. The method as in any of claims 1-16, wherein the plant is a member of the genus Spinacia.

19. The method of claim 18, wherein the plant is Spinacia oleracea.

20. The method as in any of claims 1-16, wherein the plant is selected from the group consisting of arugula, asparagus, beans, runner beans, beetroot, beet greens, brussel sprouts, cabbage, carrots, collard greens, cauliflower, celery, chives, kale, legumes, lettuce, endive, marrow, mushrooms, onions, parlsey, green peas, blueberries, potatoes, radishes, rhubarb, spinach, tomatoes, turnips, apples, apricots, bananas, gooseberries, grapefruits, melons, oranges, peaches, pears, pineapples, plums, raspberries, strawberries, bok choy, dandelion greens, escarole, cole, mache, mustard greens, radicchio, rapini, swiss chard and watercress.

21. The method of any of claims 1-16, wherein the plant is a leafy green vegetable.

22. The method of claim 4, 8, 12 or 16, wherein the nutrient quality indicators are vitamins.

23. The method of claim 4, 8, 12, or 16, wherein the nutrient quality indicators are selected from the group consisting of vitamin C, ascorbic acid, folate, folic acid, vitamin B9, β-carotene, B12, a vitamin A precursor, lutein, calcium, iron, vitamin A and vitamin E.

24. The method of claim 2, 3, 6, 7, 10, 11, 14 or 15, wherein the concentration of nitrate in the plant is reduced by 100-0.005% DW.

25. The method of claim 2, 3, 6, 7, 10, 11, 14 or 15, wherein the concentration of nitrate in the plant is reduced to zero.

26. The method of claim 1, 3, 5, 7, 9, 11, 13, or 15, wherein the concentration of oxalic acid or oxalate in the plant is reduced by 100-0.005% DW.

27. The method of claim 2, 3, 6, 7, 10, 11, 14 or 15, wherein the nitrate concentration in the plant is reduced to undetectable levels.

28. The method of claim 26, wherein the oxalic acid or oxalate concentration in the plant is reduced by 75-25% DW.

29. The method of claim 26, wherein the oxalic acid or oxalate concentration in the plant is reduced by 67-50% DW.

30. The method of any claims 1-16, wherein the plants retain a marketable appearance.

31. The method of any claims 1-16, wherein the flavor content of the plant is not altered.

32. The method of any of claims 1-16, wherein the flavor content of the plant is altered.

33. The method of any of claims 1-16, wherein the nutritional content of the plant is improved.

34. The method of any claims 1-16, wherein the nutritional content of the plant is reduced.

35. The method of any of claims 1-16, wherein the plant is a mature plant.

36. The plant produced by any of claims 1-16.

37. The plant of claim 36, wherein the concentration of nitrate is reduced.

38. The plant of claim 37, wherein the concentration of nitrate is reduced by 10%.

39. The plant of claim 37, wherein the concentration of nitrate is reduced by 20%.

40. The plant of claim 37, wherein the concentration of nitrate is reduced by 30%.

41. The plant of claim 37, wherein the concentration of nitrate is reduced by 40%.

42. The plant of claim 37, wherein the concentration of nitrate is reduced by 50%.

43. The plant of claim 37, wherein the concentration of nitrate is reduced by 60%.

44. The plant of claim 37, wherein the concentration of nitrate is reduced by 70%.

45. The plant of claim 37, wherein the concentration of nitrate is reduced by 80%.

46. The plant of claim 37, wherein the concentration of nitrate is reduced by 90%.

47. The plant of claim 37, wherein the concentration of nitrate is reduced by 100%.

48. A plant having a reduced concentration of oxalic acid or oxalate.

49. The plant of claim 48, wherein the concentration of oxalic acid or oxalate is reduced by 10%.

50. The plant of claim 48, wherein the concentration of oxalic acid or oxalate is reduced by 20%.

51. The plant of claim 48, wherein the concentration of oxalic acid or oxalate is reduced by 30%.

52. The plant of claim 48, wherein the concentration of oxalic acid or oxalate is reduced by 40%.

53. The plant of claim 48, wherein the concentration of oxalic acid or oxalate is reduced by 50%.

54. The plant of claim 48, wherein the concentration of oxalic acid or oxalate is reduced by 60%.

55. The plant of claim 48, wherein the concentration of oxalic acid or oxalate is reduced by 70%.

56. The plant of claim 48, wherein the concentration of oxalic acid or oxalate is reduced by 80%.

57. The plant of claim 48, wherein the concentration of oxalic acid or oxalate is reduced by 90%.

58. The plant of claim 48, wherein the concentration of oxalic acid or oxalate is reduced by 100%.

59. The method of any of claims 1-16, wherein the plant is selected from the group consisting of corn, rapeseed, alfalfa, rice, rye, millet, pearl millet, proso, foxtail millet, finger millet, sunflower, safflower, wheat, soybean, tobacco, potato, peanuts, cotton, potato, cassava, coffee, coconut, pineapple, cocoa, banana, avocado, fig, guava, mango, olive, papaya, cashew, macadamia, almond, sugar beets, sugarcane, oats, duckweed, barley, vegetables, ornamentals, conifers, tomatoes, green beans, lima beans, peas, Cucumis (including cucumber, cantaloupe, and musk melon), azalea, hydrangea, hibiscus, roses, tulips, daffodils, petunias, carnation, poinsettia, chrysanthemum, legumes (including peas and beans, such as guar, locust bean, fenugreek, soybean, garden beans, cowpea, mungbean, lima bean, fava bean, lentils, chickpea), peanuts, crown vetch, hairy vetch, adzuki bean, mung bean, and chickpea, lupine, trifolium, field bean, clover, Lotus, trefoil, lens, lentil, false indigo, alfalfa, orchard grass, tall fescue, perennial ryegrass, creeping bent grass, redtop grass, aneth, artichoke, blackberry, canola, cilantro, clementines, eucalyptus, fennel, grapefruit, honey dew, jicama, kiwifruit, lemon, lime, mushroom, nut, okra, orange, parsley, persimmon, plantain, pomegranate, poplar, radiata pine, Southern pine, sweetgum, tangerine, triticale, vine, yams, apple, pear, quince, cherry, apricot, melon, hemp, buckwheat, grape, raspberry, chenopodium, blueberry, nectarine, peach, plum, watermelon, eggplant, pepper, cauliflower, broccoli, onion, carrot, leek, beet, broad bean, celery, radish, pumpkin, endive, gourd, garlic, snapbean, squash, turnip, asparagas, and zuchini.
